(19) United States
(12) Patent Application Publication
(10) Pub. No.: US 2017/0165168 A1
(43) Pub. Date: Jun. 15, 2017

(54) EMULSIONS INCLUDING AT LEAST ONE SPECIAL PRESERVATIVE COMBINATION

(71) Applicant: Henkel AG & Co. KGaA, Duesseldorf (DE)

(72) Inventors: Heike Schelges, Willich (DE); Rainer Simmering, Grevenbroich (DE); Barbara Heide, Krefeld (DE); Melanie Rauschenberg, Kamen (DE)

(73) Assignee: Henkel AG & Co. KGaA, Duesseldorf (DE)

(21) Appl. No.: 15/372,161

(22) Filed: Dec. 7, 2016

(30) Foreign Application Priority Data
Dec. 11, 2015 (DE) 10 2015 225 005.9

(51) Int. Cl. A61K 8/49; A61Q 19/10; A61K 8/34; A61K 8/23; A61K 8/36; A61K 8/06; A61K 8/44

(52) U.S. Cl. CPC A61K 8/4953; A61K 8/06; A61Q 19/10; A61K 8/44; A61K 8/4926; A61K 8/23; A61K 8/36; A61K 8/34; A61K 8/361; A61K 8/347; A61K 8/345; A61K 2800/592; A61K 2800/524

(57) ABSTRACT

Cosmetic agents in the form of emulsions include synergistically effective preservative combinations. The cosmetic agents are used to cleanse and/or nurture skin and/or hair.

### FIELD OF THE INVENTION

The present invention generally relates to cosmetic agents that include at least one emulsifier, at least one oil and/or one wax and/or one ester, and at least one special preservative combination. The aforementioned cosmetic agents are suitable for cleansing skin and/or hair and have an outstanding shelf life.

Moreover, the present invention relates to the use of the cosmetic agents according to the invention to cleanse and nurture skin and/or hair.

Finally, the present invention relates to the use of special preservative combinations to preserve cleansing compositions in the form of emulsions.

### BACKGROUND OF THE INVENTION

Due to their composition, cosmetic agents can serve as a growth medium for germs and microorganisms. These germs can result in the microbial contamination of the user and alter the ingredients of the cosmetics so as to create substances having adverse effects such as sensitization or skin irritation. To avoid these undesired consequences while ensuring a certain minimum shelf life of the cosmetics, the cosmetic agents should include preservative ingredients. Since preservatives themselves may have irritative potential, the use thereof in cosmetics is strictly regulated.

The skin microflora have a crucial influence on various cosmetic parameters. For instance, pathogenic germs such as Staphylococcus aureus play a pivotal role in the formation of skin impurities. The latest studies also indicate that an imbalance in the skin microflora can have an impact on the aging of the skin, since undesired germs result in an increased immune defense which, in turn, leads to increased inflammatory reactions during the course of which skin aging markers are stimulated.

It is therefore desirable to provide preservative compositions that prevent the colonization of a cosmetic product, or of the skin, with undesired germs while not or not substantially damaging the natural skin flora.

The mixing of different antimicrobial substances in order to increase the antimicrobial activity is known in principle. For instance, WO 03/043593 A1 proposes the combination of conventional antimicrobial substances such as triclosan, phenoxyethanol or hexetidine with ethyl lauroyl arginate in order to strengthen the antibacterial effect. In WO 2007/014580 A1, preservative mixtures are proposed which, besides ethyl lauroyl arginate, include salts of organic or inorganic acids, particularly sodium citrate, sodium acetate, sodium glutamate, sodium fumarate, sodium malate, sodium gluconate, sodium laurate, sodium lactate, sodium hexametaphosphate, sodium tert-butylhydroquinone, sodium propyl paraben, or the hydrochlorides of glucosamine or ethanolamine. Cosmetic compositions that include a preservative mixture of ethyl lauroyl arginate and parabens, imidazolyl urea, phenoxyethanol, DMDM hydantoin, 2-methyl-5-chloro-3,4-isothiazolinone/2-methyl-3,4-isothiazolinone and quaternium-15 are disclosed in EP 1414394 B1.

It is therefore further desirable to provide antimicrobial compositions that are highly effective in small quantities.

Summarily, it is desirable to provide cosmetic agents having a good cleansing and nurturing effect while simultaneously having an outstanding shelf life. In particular, synergistic preservative combinations were to be identified that are highly effective in small concentrations and, due to the overall reduced quantities used, enable the manufacture of low-irritant and low-sensitizing cosmetic agents.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic agent in a cosmetically acceptable carrier includes at least one emulsifier; at least one compound selected from the group of oils, waxes, esters or mixtures thereof; at least one preservative mixture selected from the group of chloroxylenol and phenoxyisopropanol, undecylenic acid and formic acid, phenoxyisopropanol and piroctone olamine, phenoxyisopropanol and formic acid, sulfite(s) and hexetidine, ethyl lauroyl arginate and formic acid, ethyl lauroyl arginate and chloroxylenol, hexetidine and benzyl alcohol, hexetidine and chloroxylenol, hexetidine and piroctone olamine, hexetidine and chlorphenesin, hexetidine and formic acid; and mixtures thereof

At least one preservative mixture selected from the group of chloroxylenol and phenoxyisopropanol, undecylenic acid and formic acid, phenoxyisopropanol and piroctone olamine, phenoxyisopropanol and formic acid, sulfite(s) and hexetidine, ethyl lauroyl arginate and formic acid, ethyl lauroyl arginate and chloroxylenol, hexetidine and benzyl alcohol, hexetidine and chloroxylenol, hexetidine and piroctone olamine, hexetidine and chlorphenesin, hexetidine and formic acid, as well as mixtures thereof, is/are used in a method for preserving cleansing compositions in the form of emulsions.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it was found that the use of certain preservative mixtures in cosmetic agents results in a synergistic effect with respect to the preservative effect. The quantity of preservative used can thus be reduced without negatively impacting the preservative effect. Due to the reduced quantity of preservatives, the cosmetic agents according to the invention are low-irritant and low-sensitizing.

The present invention is directed to a cosmetic agent that includes, in a cosmetically acceptable carrier,
a) at least one emulsifier,
b) at least one compound selected from the group of oils, waxes, esters or mixtures thereof,
c) at least one preservative mixture selected from the group of

chloroxylenol and phenoxyisopropanol

undecylenic acid and formic acid

phenoxyisopropanol and piroctone olamine

phenoxyisopropanol and formic acid

sulfite(s) and hexetidine

ethyl lauroyl arginate and formic acid

ethyl lauroyl arginate and chloroxylenol

hexetidine and benzyl alcohol

hexetidine and chloroxylenol

hexetidine and piroctone olamine

hexetidine and chlorphenesin

hexetidine and formic acid

and mixtures thereof

The cosmetic agents of the present invention are preferably emulsions in the form of cleansing milks, impregnation solutions for cleansing wipes, and emulsions for removing eye makeup.

The term “preservative mixture” is understood according to the invention as a mixture of two of the preservatives listed above under feature b).

Moreover, the term “emulsifier” is to be understood according to the invention as amphiphilic (bifunctional) compounds consisting of at least one hydrophobic part and at least one hydrophilic molecular part. The hydrophobic group is preferably a hydrocarbon chain with 8 to 28 carbon atoms, which can be saturated or unsaturated, linear or branched. Especially preferably, this C₈-C₂₈ alkyl chain is linear.

Furthermore, the term “wax” is understood in the context of the present invention as a substance that is kneadable or solid to brittle and hard at 20° C., has a coarse to fine crystalline structure, and whose color is translucent to opaque but not glassy. Moreover, these substances melt above 25° C. without decomposing, are slightly fluid a little above the melting point (low viscosity), have a strongly temperature-dependent consistency and solubility and can be polished under slight pressure.

In addition, the term “ester” is understood according to the invention as carboxylic acid derivatives that have at least one functional group Ri—C(O)—O—R₂, where R₁ and R₂, each independently of one another, stand for C₂-C₃₀ alkyl groups, C₂-C₃₀ alkylene groups and C₂-C₃₀ aralkyl groups. Preferred esters include exactly one of the functional groups listed above. Such esters can be obtained, for example, by reacting a carboxylic acid with an alcohol.

Unless indicated otherwise, the signifier wt % refers herein to the total weight of the cosmetic agents according to the invention, with the sum of all ingredients of the agents according to the invention yielding 100 wt %.

The cosmetic agent according to the invention includes at least one emulsifier as a first essential component a).

In relation to the present invention, it has been found to be preferable if the cosmetic agent includes a nonionic emulsifier. According to the invention, “nonionic emulsifiers” are understood as referring to emulsifiers having no charged groups. “Charged groups” are to be understood both as permanently cationic and anionic groups and temporarily cationic and anionic groups. Permanently cationic and anionic groups have a cationic or anionic charge depending on their pH value. In contrast, temporarily cationic and anionic groups have a cationic or anionic charge only at certain pH values. Preferred cosmetic agents of the present invention are therefore characterized in that they include at least one wax, the wax being selected from the group of (i) coconut fatty acid glycerin mono-, di-and triesters; (ii) Butyrospermum parkii (Shea butter); (iii) esters of saturated, monovalent C₈₋₁₈ alcohols with saturated C₁₂₋₁₈ monocarboxylic acids; (iv) linear, primary C₁₂-C₂₄ alkanols; (v) esters from a saturated, monovalent C₁₆-C₆₀ alkanol and a saturated C₈-C₃₆ monocarboxylic acid, particularly cetyl behenate, stearyl behenate and C₂₀-C₄₀ alkyl stearate; (vi) glycerin triesters of saturated linear C₁₂-C₃₀ carboxylic acids, which can be hydroxylated, particularly hydrated palm oil, hydrated coconut oil, hydrated castor oil, glycerin tribehenate and glyceryltri-12-hydroxystearate; (vii) natural vegetable waxes, particularly candelilla wax, carnauba wax, Japan wax, sugar cane wax, ouricoury wax, cork wax, sunflower wax, fruit waxes; (viii) animal waxes, particularly beeswax, shellac wax and spermaceti; (ix) synthetic waxes, particularly montan ester waxes, hydrated jojoba waxes and sasol waxes, polyalkylene waxes and polyethylene glycol waxes, C₂₀-C₄₀ dialkyl esters of dimeric acids, C₃₀₋₅₀ alkyl beeswax, and alkyl and alkylaryl esters of dimerized fatty acids, paraffin waxes; as well as (x) mixtures thereof.

Advantageously, the at least one emulsifier is used in the cosmetic agents according to the invention in certain quantity ranges. Preferred cosmetic agents of the present invention are therefore characterized in that they include—with respect to their total weight—0.1 to 40 wt %, preferably 0.3 to 35 wt %, preferably 0.5 to 30 wt %, particularly 1.0 to 20 wt %, of at least one emulsifier. The use of the quantities indicated above ensures sufficient emulsification of the ingredients, thus enabling a high level of storage stability on the part of the cosmetic agents according to the invention.

As a second essential component b), the cosmetic agents according to the invention include at least one compound selected from the group of oils, waxes, esters or mixtures thereof.

It has proven advantageous in relation to the present invention if the cosmetic agents include at least one volatile non-silicone oil and/or vegetable oil. According to the invention, volatile non-silicone oils are understood as being oils which do not include any silicon atoms and which, at 20° C. and an ambient pressure of 1,013 hPa, have a vapor pressure from 2.66 to 40,000 Pa (0.02 to 300 mm Hg), preferably from 10 to 12,000 Pa (0.1 to 90 mm Hg), more preferably from 13 to 3,000 Pa (0.1 to 23 mm Hg), particularly from 15 to 500 Pa (0.1 to 4 mm Hg). It is therefore preferred according to the invention if the cosmetic agent includes at least one oil, the oil being selected from the group of (i) volatile non-silicone oils, particularly liquid paraffin oils and isoparaffin oils, such as isodecane, isoundecane, isododecane, isotridecane, isotetradecane, isopentadecane, isohexadecane and isoeicosane; (ii) vegetable oils, particularly sunflower oil, olive oil, soy oil, rapeseed oil, almond oil, jojoba oil, orange oil, wheat germ oil, peach kernel oil, and the liquid portions of coconut oil; as well as (iii) mixtures thereof. The use of the abovementioned oils in the cosmetic agents according to the invention results in an elevated nurturing effect as well as in the conditioning of the skin and/or hair.

Moreover, it is preferred in relation to the present invention if the cosmetic agents include at least one wax. Preferred cosmetic agents of the present invention are therefore characterized in that they include at least one wax, the wax being selected from the group of (i) coconut fatty acid glycerin mono-, di-and triesters; (ii) Butyrospermum parkii (Shea butter); (iii) esters of saturated, monovalent C₈₋₁₈ alcohols with saturated C12-18 monocarboxylic acids; (iv) linear, primary C₁₂-C₂₄ alkanols; (v) esters from a saturated, monovalent C₁₆-C₆₀ alkanol and a saturated C₈-C₃₆ monocarboxylic acid, particularly cetyl behenate, stearyl behenate and C₂₀-C₄₀ alkyl stearate; (vi) glycerin triesters of saturated linear C₁₂-C₃₀) carboxylic acids, which can be hydroxylated, particularly hydrated palm oil, hydrated coconut oil, hydrated castor oil, glycerin tribehenate and glyceryltri-12-hydroxystearate; (vii) natural vegetable waxes, particularly candelilla wax, carnauba wax, Japan wax, sugar cane wax, ouricoury wax, cork wax, sunflower wax, fruit waxes; (viii) animal waxes, particularly beeswax, shellac wax and spermaceti; (ix) synthetic waxes, particularly montan ester waxes, hydrated jojoba waxes and sasol waxes, polyalkylene waxes and polyethylene glycol waxes, C₂₀-C₄₀ dialkyl esters of dimeric acids, C₃₀-C₅₀ alkyl beeswax, and alkyl and alkylaryl esters of dimerized fatty acids, paraffin waxes; as well as (x) mixtures thereof. Especially preferred are commercial products with the INCI designation Cocoglycerides, particularly the commercial products Novata® (ex BASF), especially preferably Novata® AB, a mixture of C₁₂₋₁₈ mono-, di-and triglycerides that melts in the range from 30 to 32° C., and the products of the Softisan line (Sasol Germany GmbH) with INCI designation Hydrogenated Cocoglycerides, particularly Softisan 100, 133, 134, 138, 142. Other preferred esters of saturated, monovalent C₁₂₋₁₈ alcohols with saturated C₁₂₋₁₈ monocarboxylic acids are stearyl laurate, cetearyl stearate (e.g., Crodamol® CSS), cetyl palmitate (e.g., Cutina® CP) and myristyl myristate (e.g., Cetiol® MM). A C₂₀-C₄₀ alkyl stearate is also preferably used as a wax component. This ester is known by the name Kesterwachs® K82H or Kesterwachs® K80H and sold by Koster Keunen Inc.

In addition, it has proven advantageous in relation to the present invention if the cosmetic agent includes at least one ester. It is therefore preferred according to the invention if the cosmetic agent includes at least one ester, the ester being selected from the group of (i) triethyl citrates, (ii) dicarboxylic acid esters of linear or branched C₂-C₁₀ alkanols, (iii) symmetrical, asymmetrical or cyclic esters of carbonic acid with alcohols, (iv) esters of dimers of unsaturated C₁₂₋₂₂ carboxylic acids with monovalent, linear, branched and cyclic C₂₋₁₈ alkanols or C₂₋₆ alkanols, (v) benzoic acid esters of linear or branched C₈₋₂₂ alkanols, such as benzoic acid C₁₂₋₁₅ alkyl ester and benzoic acid isostearyl ester and benzoic acid octyldodecyl ester; as well as (vi) mixtures thereof. The use of the abovementioned esters also results in good nurturing and conditioning of the skin and/or hair.

Especially preferred embodiments of the present invention include at least one of the abovementioned oils and/or waxes and/or an abovementioned ester.

As a third essential component c), the cosmetic agent includes at least one specific preservative mixture. These preservative mixtures have a synergistic effect in relation to the antimicrobial effect and thus result in the especially effective preservation of the cosmetic agents according to the invention. What is more, due to the synergistic effect, the quantities used thereof can be reduced, thereby resulting in low-irritant and low-sensitizing cosmetic agents.

The cosmetic agents according to the invention preferably have a certain weight ratio of the preservatives b) included in these agents. It is therefore preferred in relation to the present invention if the cosmetic agents have a weight ratio of the first preservative to the second preservative in the preservative mixture b) from 10:1 to 1:10, preferably from 8:1 to 1:8, preferably from 5:1 to 1:5, particularly from 2:1 to 1:2. The use of such weight ratios has proven to be especially advantageous with respect to the synergistic increase in the preservative performance of this mixture.

The cosmetic agent according to the invention preferably includes the preservative mixture c) in certain quantity ranges. Preferred cosmetic agents according to the invention are therefore characterized in that they include—with respect to their total weight—0.001 to 10 wt %, preferably 0.005 to 7.0 wt %, preferably 0.01 to 4.0 wt %, particularly 0.05 to 2.0 wt %, of at least one preservative mixture (c). The quantities cited above refer to the total quantity of the preservative mixture, i.e., of the mixture of the two abovementioned preservatives. The use of such quantities of the preservative mixture results in an outstanding preservation of the cosmetic agents according to the invention. Moreover, due to the synergistic effect of the preservative mixture, the quantities used thereof are reduced without negatively impacting the preservative performance. The cosmetic agents according to the invention are therefore especially low-irritant and low-sensitizing.

Especially preferred agents K1 to K577 are listed in the tables below (all data in wt % unless indicated otherwise):

| | K1 | K2 | K3 | K4 |
|---|---|---|---|---|
| Emulsifier | 0.1-40 | 0.3-35 | 0.5-30 | 1.0-20 |
| Oil | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K5 | K6 | K7 | K8 |
| Emulsifier | 0.1-40 | 0.3-35 | 0.5-30 | 1.0-20 |
| Oil | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K9 | K10 | K11 | K12 |
| Emulsifier | 0.1-40 | 0.3-35 | 0.5-30 | 1.0-20 |
| Oil | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K13 | K14 | K15 | K16 |
| Emulsifier | 0.1-40 | 0.3-35 | 0.5-30 | 1.0-20 |
| Oil | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K17 | K18 | K19 | K20 |
| Emulsifier | 0.1-40 | 0.3-35 | 0.5-30 | 1.0-20 |
| Oil | 0.1-50 | 0.3-45 | 0.5-40 | 1.0-30 |
| Sulfite¹⁾ and hexetidine (2:1) | 0.001-10 | 0.005-7.0 | 0.01-4.0 | 0.05-2.0 |
| | K22 | K23 | K24 | K25 |
| Emulsifier | 0.1-40 | 0.3-35 | 0.5-30 | 1.0-20 |
| Oil | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K25 | K26 | K27 | K28 |
| Emulsifier | 0.1-40 | 0.3-35 | 0.5-30 | 1.0-20 |
| Oil | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K29 | K30 | K31 | K32 |
| Emulsifier | 0.1-40 | 0.3-35 | 0.5-30 | 1.0-20 |
| Oil | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K33 | K34 | K35 | K36 |
| Emulsifier | 0.1-40 | 0.3-35 | 0.5-30 | 1.0-20 |
| Oil | 0.1-50 | 0.3-45 | 0.5-40 | 1.0-30 |
| Hexetidine and chloroxylenol (1:5) | 0.001-10 | 0.005-7.0 | 0.01-4.0 | 0.05-2.0 |
| | K37 | K38 | K39 | K40 |
| Emulsifier | 0.1-40 | 0.3-35 | 0.5-30 | 1.0-20 |
| Oil | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K41 | K42 | K43 | K44 |
| Emulsifier | 0.1-40 | 0.3-35 | 0.5-30 | 1.0-20 |
| Oil | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K45 | K46 | K47 | K48 |
| Emulsifier | 0.1-40 | 0.3-35 | 0.5-30 | 1.0-20 |
| Oil | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |
| | K49 | K50 | K51 | K52 |
| Emulsifier | 0.1-40 | 0.3-35 | 0.5-30 | 1.0-20 |
| Wax | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K53 | K54 | K55 | K56 |
| Emulsifier | 0.1-40 | 0.3-35 | 0.5-30 | 1.0-20 |
| Wax | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K57 | K58 | K59 | K60 |
| Emulsifier | 0.1-40 | 0.3-35 | 0.5-30 | 1.0-20 |
| Wax | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K61 | K62 | K63 | K64 |
| Emulsifier | 0.1-40 | 0.3-35 | 0.5-30 | 1.0-20 |
| Wax | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K65 | K66 | K67 | K68 |
| Emulsifier | 0.1-40 | 0.3-35 | 0.5-30 | 1.0-20 |
| Wax | 0.1-50 | 0.3-45 | 0.5-40 | 1.0-30 |
| Sulfite¹⁾ and hexetidine (2:1) | 0.001-10 | 0.005-7.0 | 0.01-4.0 | 0.05-2.0 |
| | K69 | K70 | K71 | K72 |
| Emulsifier | 0.1-40 | 0.3-35 | 0.5-30 | 1.0-20 |
| Wax | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K73 | K74 | K75 | K76 |
| Emulsifier | 0.1-40 | 0.3-35 | 0.5-30 | 1.0-20 |
| Wax | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K77 | K78 | K79 | K80 |
| Emulsifier | 0.1-40 | 0.3-35 | 0.5-30 | 1.0-20 |
| Wax | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K81 | K82 | K83 | K84 |
| Wax | 0.5-60 | 1.0-50 | 1.5-40 | 2.0-30 |
| Hexetidine and chloroxylenol (1:5) | 0.001-10 | 0.005-7.0 | 0.01-4.0 | 0.05-2.0 |
| | K85 | K86 | K87 | K88 |
| Emulsifier | 0.1-40 | 0.3-35 | 0.5-30 | 1.0-20 |
| Wax | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K89 | K90 | K91 | K92 |
| Emulsifier | 0.1-40 | 0.3-35 | 0.5-30 | 1.0-20 |
| Wax | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K93 | K94 | K95 | K96 |
| Emulsifier | 0.1-40 | 0.3-35 | 0.5-30 | 1.0-20 |
| Wax | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |
| | K97 | K98 | K99 | K100 |
| Emulsifier | 0.1-40 | 0.3-35 | 0.5-30 | 1.0-20 |
| Ester | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K101 | K102 | K103 | K104 |
| Emulsifier | 0.1-40 | 0.3-35 | 0.5-30 | 1.0-20 |
| Ester | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K105 | K106 | K107 | K108 |
| Emulsifier | 0.1-40 | 0.3-35 | 0.5-30 | 1.0-20 |
| Ester | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K109 | K110 | K111 | K112 |
| Emulsifier | 0.1-40 | 0.3-35 | 0.5-30 | 1.0-20 |
| Ester | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K113 | K114 | K115 | K116 |
| Emulsifier | 0.1-40 | 0.3-35 | 0.5-30 | 1.0-20 |
| Ester | 0.1-50 | 0.3-45 | 0.5-40 | 1.0-30 |
| Sulfite¹⁾ and hexetidine (2:1) | 0.001-10 | 0.005-7.0 | 0.01-4.0 | 0.05-2.0 |
| | K117 | K118 | K119 | K120 |
| Emulsifier | 0.1-40 | 0.3-35 | 0.5-30 | 1.0-20 |
| Ester | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K121 | K122 | K123 | K124 |
| Emulsifier | 0.1-40 | 0.3-35 | 0.5-30 | 1.0-20 |
| Ester | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K125 | K126 | K127 | K128 |
| Emulsifier | 0.1-40 | 0.3-35 | 0.5-30 | 1.0-20 |
| Ester | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K129 | K130 | K131 | K132 |
| Emulsifier | 0.1-40 | 0.3-35 | 0.5-30 | 1.0-20 |
| Ester | 0.1-50 | 0.3-45 | 0.5-40 | 1.0-30 |
| Hexetidine and chloroxylenol (1:5) | 0.001-10 | 0.005-7.0 | 0.01-4.0 | 0.05-2.0 |
| | K133 | K134 | K135 | K136 |
| Emulsifier | 0.1-40 | 0.3-35 | 0.5-30 | 1.0-20 |
| Ester | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K137 | K138 | K139 | K140 |
| Emulsifier | 0.1-40 | 0.3-35 | 0.5-30 | 1.0-20 |
| Ester | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K141 | K142 | K143 | K144 |
| Emulsifier | 0.1-40 | 0.3-35 | 0.5-30 | 1.0-20 |
| Ester | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |
| | K145 | K146 | K147 | K148 |
| Emulsifier | 0.1-40 | 0.3-35 | 0.5-30 | 1.0-20 |
| Oil + wax | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K149 | K150 | K151 | K152 |
| Emulsifier | 0.1-40 | 0.3-35 | 0.5-30 | 1.0-20 |
| Oil + wax | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K153 | K154 | K155 | K156 |
| Emulsifier | 0.1-40 | 0.3-35 | 0.5-30 | 1.0-20 |
| Oil + wax | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K157 | K158 | K159 | K160 |
| Emulsifier | 0.1-40 | 0.3-35 | 0.5-30 | 1.0-20 |
| Oil + wax | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K161 | K162 | K163 | K164 |
| Emulsifier | 0.1-40 | 0.3-35 | 0.5-30 | 1.0-20 |
| Oil + wax | 0.1-50 | 0.3-45 | 0.5-40 | 1.0-30 |
| Sulfite¹⁾ and hexetidine (2:1) | 0.001-10 | 0.005-7.0 | 0.01-4.0 | 0.05-2.0 |
| | K165 | K166 | K1671 | K168 |
| Emulsifier | 0.1-40 | 0.3-35 | 0.5-30 | 1.0-20 |
| Oil + wax | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K169 | K170 | K171 | K172 |
| Emulsifier | 0.1-40 | 0.3-35 | 0.5-30 | 1.0-20 |
| Oil + wax | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K173 | K174 | K174 | K176 |
| Emulsifier | 0.1-40 | 0.3-35 | 0.5-30 | 1.0-20 |
| Oil + wax | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K177 | K178 | K179 | K180 |
| Emulsifier | 0.1-40 | 0.3-35 | 0.5-30 | 1.0-20 |
| Oil + wax | 0.1-50 | 0.3-45 | 0.5-40 | 1.0-30 |
| Hexetidine and chloroxylenol (1:5) | 0.001-10 | 0.005-7.0 | 0.01-4.0 | 0.05-2.0 |
| | K181 | K182 | K183 | K184 |
| Emulsifier | 0.1-40 | 0.3-35 | 0.5-30 | 1.0-20 |
| Oil + wax | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K185 | K186 | K187 | K188 |
| Emulsifier | 0.1-40 | 0.3-35 | 0.5-30 | 1.0-20 |
| Oil + wax | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K189 | K190 | K191 | K192 |
| Emulsifier | 0.1-40 | 0.3-35 | 0.5-30 | 1.0-20 |
| Oil + wax | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |
| | K193 | K194 | K195 | K196 |
| Emulsifier | 0.1-40 | 0.3-35 | 0.5-30 | 1.0-20 |
| Oil + ester | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K197 | K198 | K199 | K200 |
| Emulsifier | 0.1-40 | 0.3-35 | 0.5-30 | 1.0-20 |
| Oil + ester | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K201 | K202 | K203 | K204 |
| Emulsifier | 0.1-40 | 0.3-35 | 0.5-30 | 1.0-20 |
| Oil + ester | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K205 | K206 | K207 | K208 |
| Emulsifier | 0.1-40 | 0.3-35 | 0.5-30 | 1.0-20 |
| Oil + ester | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K209 | K210 | K211 | K212 |
| Emulsifier | 0.1-40 | 0.3-35 | 0.5-30 | 1.0-20 |
| Oil + ester | 0.1-50 | 0.3-45 | 0.5-40 | 1.0-30 |
| Sulfite¹⁾ and hexetidine (2:1) | 0.001-10 | 0.005-7.0 | 0.01-4.0 | 0.05-2.0 |
| | K213 | K214 | K215 | K216 |
| Emulsifier | 0.1-40 | 0.3-35 | 0.5-30 | 1.0-20 |
| Oil + ester | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K217 | K218 | K219 | K220 |
| Emulsifier | 0.1-40 | 0.3-35 | 0.5-30 | 1.0-20 |
| Oil + ester | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K221 | K222 | K223 | K224 |
| Emulsifier | 0.1-40 | 0.3-35 | 0.5-30 | 1.0-20 |
| Oil + ester | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K225 | K226 | K227 | K228 |
| Emulsifier | 0.1-40 | 0.3-35 | 0.5-30 | 1.0-20 |
| Oil + ester | 0.1-50 | 0.3-45 | 0.5-40 | 1.0-30 |
| Hexetidine and chloroxylenol (1:5) | 0.001-10 | 0.005-7.0 | 0.01-4.0 | 0.05-2.0 |
| | K229 | K230 | K231 | K232 |
| Emulsifier | 0.1-40 | 0.3-35 | 0.5-30 | 1.0-20 |
| Oil + ester | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K233 | K234 | K235 | K236 |
| Emulsifier | 0.1-40 | 0.3-35 | 0.5-30 | 1.0-20 |
| Oil + ester | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K237 | K238 | K239 | K240 |
| Emulsifier | 0.1-40 | 0.3-35 | 0.5-30 | 1.0-20 |
| Oil + ester | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |
| | K241 | K242 | K243 | K244 |
| Emulsifier | 0.1-40 | 0.3-35 | 0.5-30 | 1.0-20 |
| Ester + wax | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K245 | K246 | K247 | K248 |
| Emulsifier | 0.1-40 | 0.3-35 | 0.5-30 | 1.0-20 |
| Ester + wax | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K249 | K250 | K251 | K252 |
| Emulsifier | 0.1-40 | 0.3-35 | 0.5-30 | 1.0-20 |
| Ester + wax | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K253 | K254 | K255 | K256 |
| Emulsifier | 0.1-40 | 0.3-35 | 0.5-30 | 1.0-20 |
| Ester + wax | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K257 | K258 | K259 | K260 |
| Emulsifier | 0.1-40 | 0.3-35 | 0.5-30 | 1.0-20 |
| Ester + wax | 0.1-50 | 0.3-45 | 0.5-40 | 1.0-30 |
| Sulfite¹⁾ and hexetidine (2:1) | 0.001-10 | 0.005-7.0 | 0.01-4.0 | 0.05-2.0 |
| | K261 | K262 | K263 | K264 |
| Emulsifier | 0.1-40 | 0.3-35 | 0.5-30 | 1.0-20 |
| Ester + wax | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K265 | K266 | K267 | K268 |
| Emulsifier | 0.1-40 | 0.3-35 | 0.5-30 | 1.0-20 |
| Ester + wax | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K269 | K270 | K271 | K272 |
| Emulsifier | 0.1-40 | 0.3-35 | 0.5-30 | 1.0-20 |
| Ester + wax | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K273 | K274 | K275 | K276 |
| Emulsifier | 0.1-40 | 0.3-35 | 0.5-30 | 1.0-20 |
| Ester + wax | 0.1-50 | 0.3-45 | 0.5-40 | 1.0-30 |
| Hexetidine and chloroxylenol (1:5) | 0.001-10 | 0.005-7.0 | 0.01-4.0 | 0.05-2.0 |
| | K277 | K278 | K279 | K280 |
| Emulsifier | 0.1-40 | 0.3-35 | 0.5-30 | 1.0-20 |
| Ester + wax | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K281 | K282 | K283 | K284 |
| Emulsifier | 0.1-40 | 0.3-35 | 0.5-30 | 1.0-20 |
| Ester + wax | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K285 | K286 | K287 | K288 |
| Emulsifier | 0.1-40 | 0.3-35 | 0.5-30 | 1.0-20 |
| Ester + wax | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |
| | K289 | K290 | K291 | K292 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K293 | K294 | K295 | K296 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K297 | K298 | K299 | K300 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K301 | K302 | K303 | K304 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K305 | K306 | K307 | K308 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Sulfite¹⁾ and hexetidine (2:1) | 0.001-10 | 0.005-7.0 | 0.01-4.0 | 0.05-2.0 |
| | K309 | K310 | K311 | K312 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K313 | K314 | K315 | K316 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K317 | K318 | K319 | K320 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K321 | K322 | K323 | K324 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Hexetidine and chloroxylenol (1:5) | 0.001-10 | 0.005-7.0 | 0.01-4.0 | 0.05-2.0 |
| | K325 | K326 | K327 | K328 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K329 | K330 | K331 | K332 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K333 | K334 | K335 | K336 |
| Emulsifier | 0.1-40 | 0.3-35 | 0.5-30 | 1.0-20 |
| Oil + wax + ester | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |
| | K337 | K338 | K339 | K340 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K341 | K342 | K343 | K344 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K345 | K346 | K347 | K348 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K349 | K350 | K351 | K352 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K353 | K354 | K355 | K356 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Sulfite¹⁾ and hexetidine (2:1) | 0.001-10 | 0.005-7.0 | 0.01-4.0 | 0.05-2.0 |
| | K357 | K358 | K359 | K360 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K361 | K362 | K363 | K364 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K365 | K366 | K367 | K368 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K369 | K370 | K371 | K372 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Hexetidine and chloroxylenol (1:5) | 0.001-10 | 0.005-7.0 | 0.01-4.0 | 0.05-2.0 |
| | K373 | K374 | K375 | K376 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K377 | K378 | K379 | K380 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K381 | K382 | K383 | K384 |
| Emulsifier²⁾ | 0.1-40 | 0.3-35 | 0.5-30 | 1.0-20 |
| Oil³⁾ | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |
| | K385 | K386 | K387 | K388 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K389 | K390 | K391 | K392 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K393 | K394 | K395 | K396 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K397 | K398 | K399 | K400 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K401 | K402 | K403 | K404 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Sulfite¹⁾ and hexetidine (2:1) | 0.001-10 | 0.005-7.0 | 0.01-4.0 | 0.05-2.0 |
| | K405 | K406 | K407 | K408 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K409 | K410 | K411 | K412 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K413 | K414 | K415 | K416 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K417 | K418 | K419 | K420 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Hexetidine and chloroxylenol (1:5) | 0.001-10 | 0.005-7.0 | 0.01-4.0 | 0.05-2.0 |
| | K421 | K422 | K423 | K424 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K425 | K426 | K427 | K428 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K429 | K430 | K431 | K432 |
| Emulsifier²⁾ | 0.1-40 | 0.3-35 | 0.5-30 | 1.0-20 |
| Oil²⁾ + wax⁴⁾ | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |
| | K433 | K434 | K435 | K436 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K437 | K438 | K439 | K440 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K441 | K442 | K443 | K444 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K445 | K446 | K447 | K448 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K449 | K450 | K451 | K452 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Sulfite¹⁾ and hexetidine (2:1) | 0.001-10 | 0.005-7.0 | 0.01-4.0 | 0.05-2.0 |
| | K453 | K454 | K455 | K456 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K457 | K458 | K459 | K460 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K461 | K462 | K463 | K464 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K465 | K466 | K467 | K468 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Hexetidine and chloroxylenol (1:5) | 0.001-10 | 0.005-7.0 | 0.01-4.0 | 0.05-2.0 |
| | K469 | K470 | K471 | K472 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K473 | K474 | K475 | K476 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K477 | K478 | K479 | K480 |
| Emulsifier²⁾ | 0.1-40 | 0.3-35 | 0.5-30 | 1.0-20 |
| Oil²⁾ + ester⁵⁾ | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |
| | K481 | K482 | K483 | K484 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K485 | K486 | K487 | K488 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K489 | K490 | K491 | K492 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K493 | K494 | K495 | K496 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K497 | K498 | K499 | K500 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Sulfite¹ and hexetidine (2:1) | 0.001-10 | 0.005-7.0 | 0.01-4.0 | 0.05-2.0 |
| | K501 | K502 | K503 | K504 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K505 | K506 | K507 | K508 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K509 | K510 | K511 | K512 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K513 | K514 | K515 | K516 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Hexetidine and chloroxylenol (1:5) | 0.001-10 | 0.005-7.0 | 0.01-4.0 | 0.05-2.0 |
| | K517 | K518 | K519 | K520 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K521 | K522 | K523 | K524 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K525 | K526 | K527 | K528 |
| Emulsifier²⁾ | 0.1-40 | 0.3-35 | 0.5-30 | 1.0-20 |
| Wax⁴⁾ + ester⁵⁾ | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |
| | K529 | K530 | K531 | K532 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Chloroxylenol and phenoxyisopropanol | 0.001-10 | 0.005-7.0 | 0.01-4.0 | 0.05-2.0 |
| (1:2) | | | | |
| | K534 | K535 | K536 | K537 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Undecylenic acid and formic acid (1:3) | 0.001-10 | 0.005-7.0 | 0.01-4.0 | 0.05-2.0 |
| | K538 | K539 | K540 | K541 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Phenoxyisopropanol and piroctone | 0.001-10 | 0.005-7.0 | 0.01-4.0 | 0.05-2.0 |
| olamine (1:1) | | | | |
| | K542 | K543 | K544 | K545 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Phenoxyisopropanol and formic acid | 0.001-10 | 0.005-7.0 | 0.01-4.0 | 0.05-2.0 |
| (2:1) | | | | |
| | K546 | K547 | K548 | K549 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Sulfite 1) and hexetidine (2:1) | 0.001-10 | 0.005-7.0 | 0.01-4.0 | 0.05-2.0 |
| | K550 | K551 | K552 | K553 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Ethyl lauroyl arginate*HCl and formic | 0.001-10 | 0.005-7.0 | 0.01-4.0 | 0.05-2.0 |
| acid (1:2) | | | | |
| | K554 | K555 | K556 | K557 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Ethyl lauroyl arginate*HCl and | 0.001-10 | 0.005-7.0 | 0.01-4.0 | 0.05-2.0 |
| chloroxylenol (1:2) | | | | |
| | K558 | K559 | K560 | K561 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Hexetidine and benzyl alcohol (1:10) | 0.001-10 | 0.005-7.0 | 0.01-4.0 | 0.05-2.0 |
| | K562 | K563 | K564 | K565 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Hexetidine and chloroxylenol (1:5) | 0.001-10 | 0.005-7.0 | 0.01-4.0 | 0.05-2.0 |
| | K566 | K567 | K568 | K569 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Hexetidine and piroctone olamine (1:10) | 0.001-10 | 0.005-7.0 | 0.01-4.0 | 0.05-2.0 |
| | K570 | K571 | K572 | K573 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Hexetidine and chlorphenesin (1:3) | 0.001-10 | 0.005-7.0 | 0.01-4.0 | 0.05-2.0 |
| | K574 | K575 | K576 | K577 |
| Emulsifier²⁾ | 0.1-40 | 0.3-35 | 0.5-30 | 1.0-20 |
| Oil²⁾ + wax⁴⁾ + ester⁵⁾ | 0.1-50 | 0.3-45 | 0.5-40 | 1.0-30 |
| Hexetidine and formic acid (1:5) | 0.001-10 | 0.005-7.0 | 0.01-4.0 | 0.05-2.0 |

¹⁾Sulfite is selected from the group of sodium sulfite, ammonium bisulfite, ammonium sulfite, potassium sulfite, potassium hydrogen sulfite, sodium bisulfite, sodium metabisulfate and potassium metabisulfite, as well as mixtures thereof,
²⁾emulsifier selected from partial esters of polyols with 3 to 6 carbon atoms with saturated C₈-C₂₂ carboxylic acids,
³⁾oil selected from vegetable oils,
⁴⁾wax selected from linear primary C₁₂-C₂₄ alkanols,
⁵⁾ester selected from dicarboxylic acid esters of linear or branched C₂-C₁₀ alkanols.

The especially preferred cosmetic agents listed above, particularly cosmetic agents K1 to K577, have an outstanding cleansing effect and a good shelf life. By virtue of the synergistic effect of the mixture of preservatives used, the quantity thereof can be reduced without negatively impacting the preservative effect. Consequently, the cleansing compositions according to the invention are low-irritant and low-sensitizing.

The preservative effect can be further increased if the cleansing composition additionally includes at least one other preservative selected from the group of benzyl alcohol(s), chloroxylenol, phenoxyisopropanol, undecylenic acid, formic acid, piroctone olamine, sulfite(s), hexetidine, ethyl lauroyl arginate and chlorphenesin. In that case, the other preservative is selected such that it differs from the preservatives of the preservative mixture used.

Preferred cosmetic agents according to the invention are therefore characterized in that they additionally include at least one other preservative selected from the group of benzyl alcohol(s), chloroxylenol, phenoxyisopropanol, undecylenic acid, formic acid, piroctone olamine, sulfite(s), hexetidine, ethyl lauroyl arginate and chlorphenesin, provided that the cleansing composition includes at least 3 mutually different preservatives.

Other preferred cosmetic agents according to the invention are therefore characterized in that they additionally include at least two other preservatives selected from the group of benzyl alcohol(s), chloroxylenol, phenoxyisopropanol, undecylenic acid, formic acid, piroctone olamine, sulfite(s), hexetidine, ethyl lauroyl arginate and chlorphenesin, provided that the cleansing composition includes at least 4 mutually different preservatives.

Moreover, cosmetic agents are advantageous according to the invention which additionally include at least three other preservatives selected from the group of benzyl alcohol(s), chloroxylenol, phenoxyisopropanol, undecylenic acid, formic acid, piroctone olamine, sulfite(s), hexetidine, ethyl lauroyl arginate and chlorphenesin, provided that the cleansing composition includes at least 5 mutually different preservatives.

In addition, in relation to the present invention, cosmetic agents are preferred which additionally include at least four other preservatives selected from the group of benzyl alcohol(s), chloroxylenol, phenoxyisopropanol, undecylenic acid, formic acid, piroctone olamine, sulfite(s), hexetidine, ethyl lauroyl arginate and chlorphenesin, provided that the cleansing composition includes at least 6 mutually different preservatives.

Finally, cosmetic agents according to the invention are preferred which additionally include at least six other preservatives selected from the group of benzyl alcohol(s), chloroxylenol, phenoxyisopropanol, undecylenic acid, formic acid, piroctone olamine, sulfite(s), hexetidine, ethyl lauroyl arginate and chlorphenesin, provided that the cleansing composition includes at least 8 mutually different preservatives.

In this connection, it is advantageous if the at least one additional preservative is used in a certain total quantity. It is therefore preferred according to the invention if the cleansing composition includes—with respect to its total weight—0.1 to 10 wt %, preferably 0.1 to 7.0 wt %, preferably 0.1 to 4.0 wt %, particularly 0.1 to 6.0 wt %, of at least one additional preservative agent. If more than one additional preservative is used, the total quantities indicated above refer to the mixture of these preservatives. The use of such quantities of the at least one additional preservative results in an additional increase in the preservative performance without causing incompatibilities with the other ingredients of the cleansing composition according to the invention.

Especially preferred embodiments of the present invention are therefore characterized in that embodiments K1 to K577 listed above additionally include at least one other, special preservative. It is therefore especially preferred if the abovementioned embodiments K1 to K4, K49 to K52, K97 to K100, K145 to K148, K193 to K196, K241 to K244, K289 to K292, K337 to K340, K385 to K388, K433 to K436, K481 to K484 and K529 to K532 each include the following additional preservatives KM1 to KM157 or mixtures thereof in a total quantity of 0.1 to 6.0 wt %—with respect to the total weight of the cosmetic agent—(u=undecylenic acid, a=formic acid, pi=piroctone olamine, s=sulfite, h=hexetidine, e=ethyl lauroyl arginate*HCl, ch=chlorphenesin, b=benzyl alcohol).

| KM1 | KM2 | KM3 | KM4 | KM5 | KM5 | KM6 | KM7 |
|---|---|---|---|---|---|---|---|
| u | a | Pi | s | h | e | ch | u + a |
| KM8 | KM9 | KM10 | KM11 | KM12 | KM13 | KM14 | KM15 |
| u + a + pi | u + a + pi + | u + a + pi + | u + a + pi + | u + a + pi + | u + a + s¹⁾ | u + a + s¹⁾ + | u + a + s¹⁾ + |
| | s¹⁾ | s¹⁾ + h | s¹⁾ + h + e | s¹⁾ + h + e + | | h | h + e |
| | | | | ch | | | |
| KM16 | KM17 | KM18 | KM19 | KM20 | KM21 | KM22 | KM23 |
| u + a + s¹⁾ + | u + a + h | u + a + h + e | u + a + h + | u + a + e | u + a + e + | u + a + ch | u + pi |
| h + e + ch | | | e + ch | | ch | | |
| KM24 | KM25 | KM26 | KM27 | KM28 | KM29 | KM30 | KM31 |
| u + pi + s¹⁾ | u + pi + | u + pi + s¹⁾ + | u + pi + | u + pi + h | u + pi + h + | u + pi + h + | u + pi + e |
| | s¹⁾ + h | h + e | s¹⁾ + | | e | e + ch | |
| | | | h + e + ch | | | | |
| KM32 | KM33 | KM34 | KM35 | KM36 | KM37 | KM38 | KM39 |
| u + pi + e + | u + pi + ch | u + s¹⁾ | u + s¹⁾ + h | u + s¹⁾ + h + | u + s¹⁾ + h + | u + s¹⁾ + e | u + s¹⁾ + e + |
| ch | | | | e | e + ch | | ch |
| KM40 | KM41 | KM42 | KM43 | KM44 | KM45 | KM46 | KM47 |
| u + s¹⁾ + ch | u + h | u + h + e | u + h + e + | u + h + ch | u + e | u + e + ch | u + ch |
| | | | ch | | | | |
| KM48 | KM49 | KM50 | KM51 | KM52 | KM53 | KM54 | KM55 |
| a + pi | a + pi + s | a + pi + | a + pi + | a + pi + | a + pi + h | a + pi + h + | a + pi + h + |
| | | s¹⁾ + h | s¹⁾ + h + e | s¹⁾ + | | e | e + ch |
| | | | | h + e + ch | | | |
| KM56 | KM57 | KM58 | KM59 | KM60 | KM61 | KM62 | KM63 |
| a + pi + e | a + pi + e + | a + pi + ch | a + s¹⁾ | a + s¹⁾ + h | a + s¹⁾ + | a + s¹⁾ + h + | a + s¹⁾ + e |
| | ch | | | | h + e | e + ch | |
| KM64 | KM65 | KM66 | KM67 | KM68 | KM69 | KM70 | KM71 |
| a + s¹⁾ + e + | a + s¹⁾ + ch | a + h | a + h + e | a + h + e + c | a + h + ch | a + e | a + e + ch |
| ch | | | | h | | | |
| KM72 | KM73 | KM74 | KM75 | KM76 | KM78 | KM79 | KM80 |
| a + ch | pi + s¹⁾ | pi + s¹⁾ + h | pi + s¹⁾ + | pi + s¹⁾ + h + | pi + s¹⁾ + e | pi + s¹⁾ + e + | pi + s¹⁾ + |
| | | | h + e | e + ch | | ch | ch |
| KM81 | KM82 | KM83 | KM84 | KM85 | KM86 | KM87 | KM88 |
| pi + h | pi + h + e | pi + h + e + | pi + h + ch | pi + e | pi + e + ch | pi + ch | s¹⁾ + h |
| | | ch | | | | | |
| KM89 | KM90 | KM91 | KM92 | KM93 | KM94 | KM95 | KM96 |
| s¹⁾ + h + e | s¹⁾ + h + e + | s¹⁾ + h + ch | s¹⁾ + e | s¹⁾ + e + ch | s¹⁾ + ch | h + e | h + e + ch |
| | ch | | | | | | |
| KM97 | KM98 | KM99 | KM100 | KM101 | KM102 | KM103 | KM104 |
| h + ch | e + ch | u + a + b | u + a + ch + | u + a + e + | u + a + h + | u + a + s¹⁾ + | u + a + pi + |
| | | | b | ch + b | e + ch + b | h + e + ch + | s¹⁾ + |
| | | | | | | b | h + e + ch + |
| | | | | | | | b |
| KM105 | KM106 | KM107 | KM108 | KM109 | KM110 | KM111 | KM112 |
| b | u + pi + b | u + pi + | u + pi + e + | u + pi + | u + pi + | u + s¹⁾ + b | u + s¹⁾ + |
| | | ch + b | ch + b | h + e + | s¹⁾ + h + | | ch + b |
| | | | | ch + b | e + ch + | | |
| | | | | | b | | |
| KM113 | KM114 | KM115 | KM116 | KM117 | KM118 | KM119 | KM120 |
| u + s¹⁾ + e + | u + s¹⁾ + | u + h + b | u + h + | u + h + e + | u + b | u + ch + b | u + e + |
| ch + b | h + e + | | ch + b | ch + b | | | ch + b |
| | ch + b | | | | | | |
| KM121 | KM122 | KM123 | KM124 | KM125 | KM126 | KM127 | KM128 |
| a + pi + b | a + pi + | a + pi + e + | a + pi + | a + pi + | a + s¹⁾ + b | a + s¹⁾ + | a + s¹⁾ + e + |
| | ch + b | ch + b | h + e + | s¹⁾ + h + | | ch + b | ch + b |
| | | | ch + b | e + ch + | | | |
| | | | | b | | | |
| KM129 | KM130 | KM131 | KM132 | KM133 | KM134 | KM135 | KM136 |
| a + s¹⁾ + h + | a + h + b | a + h + | a + h + e + | a + b | a + ch + b | a + e + ch + | pi + s¹⁾ + b |
| e + ch + b | | ch + b | ch + b | | | b | |
| KM137 | KM138 | KM139 | KM140 | KM141 | KM142 | KM143 | KM144 |
| pi + s¹⁾ + c | pi + s¹⁾ + | pi + s¹⁾ + | pi + h + b | pi + h + | pi + h + e + | pi + b | pi + ch + b |
| h + b | e + ch + | h + e + | | ch + b | ch + b | | |
| | b | ch + b | | | | | |
| KM145 | KM146 | KM147 | KM148 | KM149 | KM150 | KM151 | KM152 |
| pi + e + | s¹⁾ + h + b | s¹⁾ + h + ch + | s¹⁾ + h + | s¹⁾ + b | s¹⁾ + ch + b | s¹⁾ + e + | h + b |
| ch + b | | b | e + ch + b | | | ch + b | |
| KM153 | KM154 | KM155 | KM156 | KM157 | | | |
| h + ch + b | h + e + | e + b | e + ch + b | ch + b | | | |
| | ch + b | | | | | | |

¹⁾Sulfite is selected from the group of sodium sulfite, ammonium bisulfite, ammonium sulfite, potassium sulfite, potassium hydrogen sulfite, sodium bisulfite, sodium metabisulfate and potassium metabisulfite, as well as mixtures thereof

Furthermore, especially preferred embodiments of the present invention are characterized in that the abovementioned cosmetic agents K5 to K8, K53 to K56, K101 to K104, K149 to K152, K197 to K200, K245 to K248, K293 to K296, K341 to K344, K389 to K392, K437 to K440, K485 to K488 and K534 to K537 each additionally include the abovementioned preservatives KM1 to KM157 or mixtures thereof in a total quantity of 0.1 to 6.0 wt %—with respect to the total weight of the cosmetic agents—, provided that, in the abovementioned KM1 to KM157, u=undecylenic acid is replaced by c=chloroxylenol and a=formic acid is replaced by p=phenoxyisopropanol.

Moreover, compositions are preferred in relation to the present invention in which the abovementioned cosmetic agents K9 to K12, K57 to K60, K105 to K108, K153 to K156, K201 to K204, K249 to K252, K297 to K300, K345 to K348, K393 to K396, K441 to K444, K489 to K492 and K538 to K541 each additionally include the abovementioned preservatives KM1 to KM157 or mixtures thereof in a total quantity of 0.1 to 6.0 wt %—with respect to the total weight of the cosmetic agents—, provided that, in the abovementioned KM1 to KM157, p=piroctone olamine is replaced by c=chloroxylenol.

In addition, compositions are preferred in relation to the present invention in which the abovementioned cosmetic agents K13 to K16, K61 to K64, K109 to K112, K157 to K160, K205 to K208, K253 to K256, K301 to K304, K349 to K352, K397 to K400, K445 to K448, K493 to K496 and K542 to K545 each additionally include the abovementioned preservatives KM1 to KM157 or mixtures thereof in a total quantity of 0.1 to 6.0 wt %—with respect to the total weight of the cosmetic agents—, provided that, in the abovementioned KM1 to KM157, a=formic acid is replaced by c=chloroxylenol.

Moreover, compositions are preferred in relation to the present invention in which the abovementioned cosmetic agents K17 to K20, K65 to K68, K113 to K116, K161 to K164, K209 to K212, K257 to K260, K305 to K308, K353 to K356, K401 to K404, K449 to K452, K497 to K500 and K546 to K549 each additionally include the abovementioned preservatives KM1 to KM157 or mixtures thereof in a total quantity of 0.1 to 6.0 wt %—with respect to the total weight of the cosmetic agents—, provided that, in the abovementioned KM1 to KM157, s=sulfite is replaced by c=chloroxylenol and h=hexetidine is replaced by p=phenoxyisopropanol.

Furthermore, it is advantageous in relation to the present invention if the abovementioned cosmetic agents K21 to K24, K69 to K72, K117 to K120, K165 to K168, K213 to K216, K261 to K264, K309 to K312, K357 to K360, K405 to K408, K453 to K456, K501 to K504 and K550 to K553 each additionally include the abovementioned preservatives KM1 to KM157 or mixtures thereof in a total quantity of 0.1 to 6.0 wt %—with respect to the total weight of the cosmetic agents—, provided that, in the abovementioned KM1 to KM157, e=ethyl lauroyl arginate*HCl is replaced by c=chloroxylenol and a=formic acid is replaced by p=phenoxyisopropanol.

Moreover, it is advantageous in relation to the present invention if the abovementioned cosmetic agents K25 to K28, K73 to K76, K121 to K124, K169 to K172, K217 to K220, K265 to K268, K313 to K316, K361 to K364, K409 to K412, K457 to K460, K505 to K508 and K554 to K557 each additionally include the abovementioned preservatives KM1 to KM157 or mixtures thereof in a total quantity of 0.1 to 6.0 wt %—with respect to the total weight of the cosmetic agents—, provided that, in the abovementioned KM1 to KM157, e=ethyl lauroyl arginate*HCl is replaced by p=phenoxyethanol.

In addition, it is advantageous in relation to the present invention if the abovementioned cosmetic agents K29 to K32, K77 to K80, K125 to K128, K173 to K176, K221 to K224, K269 to K272, K317 to K320, K365 to K368, K413 to K416, K461 to K464, K509 to K512 and K558 to K561 each additionally include the abovementioned preservatives KM1 to KM157 or mixtures thereof in a total quantity of 0.1 to 6.0 wt %—with respect to the total weight of the cosmetic agents—, provided that, in the abovementioned KM1 to KM157, h=hexetidine is replaced by p=phenoxyethanol and b=benzyl alcohol is replaced by p=phenoxyisopropanol.

Furthermore, especially preferred embodiments of the present invention are characterized in that the abovementioned cosmetic agents K33 to K36, K81 to K84, K129 to K132, K177 to K180, K225 to K228, K273 to K276, K321 to K324, K369 to K372, K417 to K420, K465 to K468, K513 to K516 and K562 to K565 each additionally include the abovementioned preservatives KM1 to KM157 or mixtures thereof in a total quantity of 0.1 to 6.0 wt %—with respect to the total weight of the cosmetic agents—, provided that, in the abovementioned KM1 to KM157, h=hexetidine is replaced by p=phenoxyisopropanol.

Moreover, compositions are preferred in relation to the present invention in which the abovementioned cosmetic agents K37 to K40, K85 to K88, K133 to K136, K181 to K184, K229 to K232, K277 to K280, K325 to K328, K373 to K376, K421 to K424, K469 to K472, K517 to K520 and K566 to K569 each additionally include the abovementioned preservatives KM1 to KM157 or mixtures thereof in a total quantity of 0.1 to 6.0 wt %—with respect to the total weight of the cosmetic agents—, provided that, in the abovementioned KM1 to KM157, h=hexetidine is replaced by c=chloroxylenol and pi=piroctone olamine is replaced by phenoxyisopropanol.

In addition, compositions are preferred in relation to the present invention in which the abovementioned cosmetic agents K41 to K44, K89 to K92, K137 to K140, K185 to K188, K233 to K236, K281 to K284, K329 to K332, K377 to K380, K425 to K428, K473 to K476, K521 to K524 and K570 to K573 each additionally include the abovementioned preservatives KM1 to KM157 or mixtures thereof in a total quantity of 0.1 to 6.0 wt %—with respect to the total weight of the cosmetic agents—, provided that, in the abovementioned KM1 to KM157, h=hexetidine is replaced by p=phenoxyisopropanol.

Finally, compositions are preferred in relation to the present invention in which the abovementioned cosmetic agents K45 to K48, K93 to K96, K141 to K144, K189 to K192, K237 to K240, K285 to K288, K333 to K336, K381 to K384, K429 to K432, K477 to K480, K525 to K528 and K574 to K577 each additionally include the abovementioned preservatives KM1 to KM157 or mixtures thereof in a total quantity of 0.1 to 6.0 wt %—with respect to the total weight of the cosmetic agents—, provided that, in the abovementioned KM1 to KM157, h=hexetidine is replaced by p=phenoxyisopropanol and a=formic acid is replaced by c=chloroxylenol.

The addition of the at least one other preservative results in a further increase in the preservative performance. What is more, the lather quality and stability is not negatively impacted and can even be improved through the addition of the at least one additional preservative.

Besides the components a) to c) that are required according to the invention, the cosmetic agents according to the invention can, in principle, include any other component known to a person skilled in the art for such cosmetic compositions. Additional active substances, adjuvants and additives are, for example:
Thickeners such as gelatins or vegetable gums, such as agar-agar, guar gum, alginates, xanthan gum, gum arabic, karaya gum, locust bean gum, flaxseed gums, dextranes, cellulose derivatives, e.g., methyl cellulose, hydroxyalkyl cellulose and carboxymethyl cellulose, starch fractions and derivatives such as amylose, amylopectin and dextrins, fully synthetic hydrocolloids such as polyvinyl alcohol,
structurants such as maleic acid and lactic acid,
solvents and solubilizers such as ethanol, isopropanol, ethylene glycol, propylene glycol, glycerin and diethylene glycol,
fiber structure-improving active substances, particularly mono-, di-and oligosaccharides such as, for example, glucose, galactose, fructose, fruit sugar and lactose,
dues for coloring the agent,
substances for setting the pH value, such as a-and 13-hydroxycarboxylic acids,
active substances such as allantoin and bisabolol,
complexing agents such as EDTA, NTA and μ-alanine diacetic acid and phosphonic acids,
ceramides. Ceramides are understood as being N-acylsphingosine (fatty acid amides of sphingosines) or synthetic analogs of such lipids (so-called pseudo-ceramides),
opacifiers such as latex, styrene/PVP and styrene/acrylamide copolymers,
pearlescent agents such as ethylene glycol mono-and disearate as well as PEG-3 distearate,
pigments,
propellants such as propane/butane mixtures, N₂O, dimethyl ether, CO₂ and air,
viscosity control agents such as salts (NaCl),
anionic, cationic and amphoteric surfactants,
cationic, nonionic and amphoteric polymers,
vitamins, particularly from the groups A, B, C, E, F and H,
UV filters, particularly benzophenones, p-aminobenzoic acid esters, diphenylacrylic acid esters, cinnamic acid esters, salicylic acid esters, benzimidazoles and o-aminobenzoic acid esters,
protein hydrolysates and cationized protein hydrolysates,
humectants or penetrating agents and/or swelling agents, particularly urea and urea derivatives, guanidine and derivatives thereof, arginine and derivatives thereof, water glass, imidazole and derivatives thereof, histidine and derivatives thereof, benzyl alcohol, glycol ether, propylene glycol ether, for example propylene glycol monoethyl ether, carbonates, hydrogen carbonates, 1,2-diols and 1,3-diols,
plant extracts, for example from green tea, white tea, oak bark, stinging nettle, Hamamelis, hops, chamomile, burdock root, horsetail, whitethorn, lime blossoms, lychee, almond, aloe vera, spruce needle, horse chestnut, sandalwood, juniper, coconut, mango, apricot, lime, wheat, kiwi, cantaloupe, orange, grapefruit, salvia, rosemary, birch, mallow, lady's smock, creeping thyme, yarrow, thyme, Melissa, restharrow, coltsfoot, marshmallow, ginseng, ginger root, Echinacea purpurea, Olea europea, Foeniculum vulgaris and Apim graveolens,
silicone oils, particularly polyalkyl siloxanes, polyaryl siloxanes and polyalkylaryl siloxanes, which can be optionally functionalized with organic groups and/or ethoxy and/or propoxy groups.

The abovementioned other ingredients can be included—with respect to the total weight of the cosmetic agent—in a total quantity of 0.001 to 50 wt %, preferably 0.01 to 40 wt %, more preferably 0.1 to 30 wt %, particularly 0.5 to 20 wt %.

A second object of the present invention is the use of the cosmetic agents according to the invention for cleansing and nurturing skin and hair.

The remarks made above in relation to the cosmetic agents according to the invention apply mutatis mutandis with respect to other preferred embodiments of the use according to the invention.

Finally, a third object of the present invention is the use of at least one preservative mixture selected from the group of chloroxylenol and phenoxyisopropanol, undecylenic acid and formic acid, phenoxyisopropanol and piroctone olamine, phenoxyisopropanol and formic acid, sulfite(s) and hexetidine, ethyl lauroyl arginate and formic acid, ethyl lauroyl arginate and chloroxylenol, hexetidine and benzyl alcohol, hexetidine and chloroxylenol, hexetidine and piroctone olamine, hexetidine and chlorphenesin, hexetidine and formic acid, as well as mixtures thereof, for preserving cleansing compositions in the form of emulsions.

The use of the abovementioned preservative mixtures results in the outstanding preservation of emulsions. By virtue of the synergistic effect of these combinations in relation to the preservative effect, the quantity of preservatives can be reduced, thereby resulting in low-irritant and low-sensitizing cleansing compositions. Emulsions are understood as being O/W emulsions, W/O emulsions and O/W/O and W/O/W emulsions.

In this connection, it can also be preferred to use an additional preservative besides the at least one preservative mixture. The preservatives KM1 to KM157 and mixtures thereof described in connection with the first object of the invention in particular are preferred.

As regards other embodiments of the method according to the invention, the remarks concerning the cleansing composition according to the invention apply mutatis mutandis.

The following examples explain the present invention without limiting it:

### EXAMPLES

The following cleansing emulsions were prepared:

Cleansing milk (data in wt %)

| Raw material | 1.1 | 1.2 | 1.3 | 1.4 | 1.5 | 1.6 |
|---|---|---|---|---|---|---|
| Helianthus annuus seed oil | 5.00 | 5.00 | 5.00 | 5.00 | 5.00 | 5.00 |
| Isopropyl palmitate | 4.98 | 4.98 | 4.98 | 4.98 | 4.98 | 4.98 |
| Rosa damascena flower water | 4.97 | 4.97 | 4.97 | 4.97 | 4.97 | 4.97 |
| Ethyl lauroyl arginate*HCl | 0.50 | — | — | — | — | 0.40 |
| Piroctone olamine | 0.40 | — | — | — | — | 1.00 |
| Sodium sulfite | — | 0.20 | — | — | — | 0.20 |
| Hexetidine | — | 0.10 | — | — | — | 0.10 |
| Formic acid | — | — | 0.50 | — | — | 0.50 |
| Phenoxyisopropanol | — | — | 1.00 | — | — | 1.00 |
| Undecylenic acid | — | — | — | — | — | 0.20 |
| Chloroxylenol | — | — | — | — | — | 0.50 |
| Benzyl alcohol | — | — | — | — | — | 1.00 |
| Chlorphenesin | — | — | — | — | — | 0.30 |
| KM ¹⁾ | — | — | — | 0.5 | 2.0 | — |
| Cetearyl alcohol | 2.50 | 2.50 | 2.50 | 2.50 | 2.50 | 2.50 |
| Cocoglycerides | 2.00 | 2.00 | 2.00 | 2.00 | 2.00 | 2.00 |
| Laurus nobilis leaf extract | 1.99 | 1.99 | 1.99 | 1.99 | 1.99 | 1.99 |
| Hydrogenated vegetable oil | 1.00 | 1.00 | 1.00 | 1.00 | 1.00 | 1.00 |
| Xanthan gum | 1.00 | 1.00 | 1.00 | 1.00 | 1.00 | 1.00 |
| Lauryl glucoside | 0.95 | 0.95 | 0.95 | 0.95 | 0.95 | 0.95 |
| Polyglyceryl-2 | 0.95 | 0.95 | 0.95 | 0.95 | 0.95 | 0.95 |
| dipolyhydroxystearate | | | | | | |
| Panthenol | 0.50 | 0.50 | 0.50 | 0.50 | 0.50 | 0.50 |
| Citric acid | 0.21 | 0.21 | 0.21 | 0.21 | 0.21 | 0.21 |
| Sodium stearoyl glutamate | 0.10 | 0.10 | 0.10 | 0.10 | 0.10 | 0.10 |
| Tocopheryl acetate | 0.09 | 0.09 | 0.09 | 0.09 | 0.09 | 0.09 |
| Citric acid | 0.02 | 0.02 | 0.02 | 0.02 | 0.02 | 0.02 |
| Aqua | up to | up to | up to | up to | up to | up to |
| | 100 | 100 | 100 | 100 | 100 | 100 |
| Raw material | 2.1 | 2.2 | 2.3 | 2.4 | 2.5 | 2.6 |
| Paraffinum liquidum | 20.00 | 20.00 | 20.00 | 20.00 | 20.00 | 20.00 |
| Glycerin | 1.99 | 1.99 | 1.99 | 1.99 | 1.99 | 1.99 |
| Ceteareth-12 | 1.39 | 1.39 | 1.39 | 1.39 | 1.39 | 1.39 |
| Triceteareth-4 phosphate | 1.27 | 1.27 | 1.27 | 1.27 | 1.27 | 1.27 |
| Cetearyl alcohol | 0.75 | 0.75 | 0.75 | 0.75 | 0.75 | 0.75 |
| Ethyl lauroyl arginate*HCl | 0.40 | — | — | — | — | 0.40 |
| Piroctone olamine | 1.00 | — | — | — | — | 1.00 |
| Sodium sulfite | — | — | — | — | — | 0.20 |
| Hexetidine | — | — | — | — | — | 0.10 |
| Formic acid | — | — | 0.50 | — | — | 0.50 |
| Phenoxyisopropanol | — | 1.00 | — | — | — | 1.00 |
| Undecylenic acid | — | — | 0.20 | — | — | 0.20 |
| Chloroxylenol | — | 0.50 | — | — | — | 0.50 |
| Benzyl alcohol | — | — | — | — | — | 1.00 |
| Chlorphenesin | — | — | — | — | — | 0.30 |
| KM ¹⁾ | — | — | — | 0.5 | 2.0 | — |
| Sorbitol | 0.70 | 0.70 | 0.70 | 0.70 | 0.70 | 0.70 |
| Panthenol | 0.50 | 0.50 | 0.50 | 0.50 | 0.50 | 0.50 |
| Carbomer | 0.30 | 0.30 | 0.30 | 0.30 | 0.30 | 0.30 |
| Perfume | 0.14 | 0.14 | 0.14 | 0.14 | 0.14 | 0.14 |
| Argania spinosa kernel oil | 0.10 | 0.10 | 0.10 | 0.10 | 0.10 | 0.10 |
| Xanthan gum | 0.09 | 0.09 | 0.09 | 0.09 | 0.09 | 0.09 |
| Trisodium dicarboxymethyl | 0.04 | 0.04 | 0.04 | 0.04 | 0.04 | 0.04 |
| alaninate | | | | | | |
| Pantolactone | 0.01 | 0.01 | 0.01 | 0.01 | 0.01 | 0.01 |
| Aqua | up to | up to | up to | up to | up to | up to |
| | 100 | 100 | 100 | 100 | 100 | 100 |

¹⁾ Preservative mixture selected from at least one of the preservative mixtures KM1 to KM157 listed above.

Cleansing milk (data in wt %)

| Raw material | 3.1 | 3.2 | 3.3 | 3.4 | 3.5 | 3.6 |
|---|---|---|---|---|---|---|
| Ethylhexyl palmitate | 6.00 | 6.00 | 6.00 | 6.00 | 6.00 | 6.00 |
| Hexanediol | 6.00 | 6.00 | 6.00 | 6.00 | 6.00 | 6.00 |
| Hexyl laurate | 3.00 | 3.00 | 3.00 | 3.00 | 3.00 | 3.00 |
| Myristyl myristate | 2.91 | 2.91 | 2.91 | 2.91 | 2.91 | 2.91 |
| Caprylic/capric triglyceride | 2.00 | 2.00 | 2.00 | 2.00 | 2.00 | 2.00 |
| Sucrose stearate | 1.91 | 1.91 | 1.91 | 1.91 | 1.91 | 1.91 |
| CI 77891 | 0.60 | 0.60 | 0.60 | 0.60 | 0.60 | 0.60 |
| Panthenol | 0.50 | 0.50 | 0.50 | 0.50 | 0.50 | 0.50 |
| Cetearyl alcohol | 0.50 | 0.50 | 0.50 | 0.50 | 0.50 | 0.50 |
| Ethyl lauroyl arginate*HCl | 0.40 | — | 0.40 | — | — | 0.40 |
| Piroctone olamine | — | 1.00 | — | — | — | 1.00 |
| Sodium sulfite | — | — | — | — | — | 0.20 |
| Hexetidine | — | — | — | — | — | 0.10 |
| Formic acid | 0.50 | — | 0.50 | 0.50 | — | 0.50 |
| Phenoxyisopropanol | — | 1.00 | — | — | 1.00 | 1.00 |
| Undecylenic acid | — | — | — | 0.20 | — | 0.20 |
| Chloroxylenol | — | — | — | — | 0.50 | 0.50 |
| Benzyl alcohol | — | — | — | — | — | 1.00 |
| Chlorphenesin | — | — | — | — | — | 0.30 |
| KM ¹⁾ | — | — | — | 0.2 | 1.5 | — |
| Glyceryl stearate | 0.50 | 0.50 | 0.50 | 0.50 | 0.50 | 0.50 |
| Cera alba | 0.50 | 0.50 | 0.50 | 0.50 | 0.50 | 0.50 |
| Perfume | 0.34 | 0.34 | 0.34 | 0.34 | 0.34 | 0.34 |
| Tocopheryl acetate | 0.30 | 0.30 | 0.30 | 0.30 | 0.30 | 0.30 |
| Hydroxypropyl methyl cellulose | 0.20 | 0.20 | 0.20 | 0.20 | 0.20 | 0.20 |
| Carbomer | 0.17 | 0.17 | 0.17 | 0.17 | 0.17 | 0.17 |
| Bisabolol | 0.09 | 0.09 | 0.09 | 0.09 | 0.09 | 0.09 |
| Myristyl alcohol | 0.09 | 0.09 | 0.09 | 0.09 | 0.09 | 0.09 |
| Sodium hydroxide | 0.06 | 0.06 | 0.06 | 0.06 | 0.06 | 0.06 |
| Retinyl palmitate | 0.06 | 0.06 | 0.06 | 0.06 | 0.06 | 0.06 |
| Arachis hypogaea oil | 0.04 | 0.04 | 0.04 | 0.04 | 0.04 | 0.04 |
| Stearic acid | 0.03 | 0.03 | 0.03 | 0.03 | 0.03 | 0.03 |
| Tocopherol | 0.03 | 0.03 | 0.03 | 0.03 | 0.03 | 0.03 |
| Hydrogenated palm glycerides | 0.02 | 0.02 | 0.02 | 0.02 | 0.02 | 0.02 |
| citrate | | | | | | |
| Sucrose | 0.02 | 0.02 | 0.02 | 0.02 | 0.02 | 0.02 |
| Aqua | up to | up to | up to | up to | up to | up to |
| | 100 | 100 | 100 | 100 | 100 | 100 |

¹⁾ Preservative mixture selected from at least one of the preservative mixtures KM1 to KM466 listed above.

Washing cream (data in wt %)

| Raw material | 4.1 | 4.2 | 4.3 | 4.4 | 4.5 | 4.6 |
|---|---|---|---|---|---|---|
| Coco-caprylate/caprate | 5.00 | 5.00 | 5.00 | 5.00 | 5.00 | 5.00 |
| Dicaprylyl carbonate | 5.00 | 5.00 | 5.00 | 5.00 | 5.00 | 5.00 |
| Pentaerythrityl distearate | 1.00 | 1.00 | 1.00 | 1.00 | 1.00 | 1.00 |
| Propylene glycol | 0.84 | 0.84 | 0.84 | 0.84 | 0.84 | 0.84 |
| Sodium polyacrylate | 0.50 | 0.50 | 0.50 | 0.50 | 0.50 | 0.50 |
| Ethyl lauroyl arginate*HCl | 0.40 | — | — | — | — | 0.40 |
| Piroctone olamine | — | — | — | — | — | 1.00 |
| Sodium sulfite | — | — | — | — | — | 0.20 |
| Hexetidine | — | 0.10 | 0.10 | | | 0.10 |
| Formic acid | 0.40 | — | — | — | — | 0.50 |
| Phenoxyisopropanol | — | — | — | — | — | 1.00 |
| Undecylenic acid | — | — | — | — | — | 0.20 |
| Chloroxylenol | — | — | 0.50 | — | — | 0.50 |
| Benzyl alcohol | — | 1.00 | — | — | — | 1.00 |
| Chlorphenesin | — | — | — | — | — | 0.30 |
| KM ¹⁾ | — | — | — | 0.1 | 1.0 | — |
| Perfume | 0.32 | 0.32 | 0.32 | 0.32 | 0.32 | 0.32 |
| Tetrasodium EDTA | 0.10 | 0.10 | 0.10 | 0.10 | 0.10 | 0.10 |
| Butylene glycol | 0.09 | 0.09 | 0.09 | 0.09 | 0.09 | 0.09 |
| Pentylene glycol | 0.04 | 0.04 | 0.04 | 0.04 | 0.04 | 0.04 |
| Hydrolyzed soy protein | 0.03 | 0.03 | 0.03 | 0.03 | 0.03 | 0.03 |
| Prunus persica fruit extract | 0.02 | 0.02 | 0.02 | 0.02 | 0.02 | 0.02 |
| Panax ginseng root extract | 0.01 | 0.01 | 0.01 | 0.01 | 0.01 | 0.01 |
| Tocopherol | 0.00 | 0.00 | 0.00 | 0.00 | 0.00 | 0.00 |
| Sebacic acid | 0.00 | 0.00 | 0.00 | 0.00 | 0.00 | 0.00 |
| 10-hydroxydecanoic acid | 0.00 | 0.00 | 0.00 | 0.00 | 0.00 | 0.00 |
| 1.10-decane diol | 0.00 | 0.00 | 0.00 | 0.00 | 0.00 | 0.00 |
| 2,4-dimethyl-3-cyclohexene | 0.00 | 0.00 | 0.00 | 0.00 | 0.00 | 0.00 |
| carboxaldehyde | | | | | | |
| Pinus sylvestris bark extract | 0.00 | 0.00 | 0.00 | 0.00 | 0.00 | 0.00 |
| Ribes nigrum leaf extract | 0.00 | 0.00 | 0.00 | 0.00 | 0.00 | 0.00 |
| Aqua | up to | up to | up to | up to | up to | up to |
| | 100 | 100 | 100 | 100 | 100 | 100 |

¹⁾ Preservative mixture selected from at least one of the preservative mixtures KM1 to KM157 listed above.

Cleansing milk cold production (data in wt %)

| Raw material | 5.1 | 5.2 | 5.3 | 5.4 | 5.5 | 5.6 |
|---|---|---|---|---|---|---|
| Glycerin | 4.98 | 4.98 | 4.98 | 4.98 | 4.98 | 4.98 |
| Caprylic/capric triglyceride | 4.26 | 4.26 | 4.26 | 4.26 | 4.26 | 4.26 |
| Ethylhexyl palmitate | 4.00 | 4.00 | 4.00 | 4.00 | 4.00 | 4.00 |
| Carthamus tinctorius seed oil | 3.00 | 3.00 | 3.00 | 3.00 | 3.00 | 3.00 |
| Cocoglycerides | 3.00 | 3.00 | 3.00 | 3.00 | 3.00 | 3.00 |
| Sorbitol | 2.10 | 2.10 | 2.10 | 2.10 | 2.10 | 2.10 |
| Glyceryl oleate citrate | 1.24 | 1.24 | 1.24 | 1.24 | 1.24 | 1.24 |
| Aluminum starch | 0.93 | 0.93 | 0.93 | 0.93 | 0.93 | 0.93 |
| octenylsuccinate | | | | | | |
| Dimethicone | 0.50 | 0.50 | 0.50 | 0.50 | 0.50 | 0.50 |
| Carbomer | 0.30 | 0.30 | 0.30 | 0.30 | 0.30 | 0.30 |
| Perfume | 0.28 | 0.28 | 0.28 | 0.28 | 0.28 | 0.28 |
| Sodium acrylate/sodium | 0.19 | 0.19 | 0.19 | 0.19 | 0.19 | 0.19 |
| acryloyldimethyl taurate | | | | | | |
| copolymer | | | | | | |
| Trilinolein | 0.16 | 0.16 | 0.16 | 0.16 | 0.16 | 0.16 |
| Polyisobutene | 0.14 | 0.14 | 0.14 | 0.14 | 0.14 | 0.14 |
| Ethyl lauroyl arginate*HCl | — | — | — | — | — | 0.40 |
| Piroctone olamine | 0.50 | 0.50 | — | — | — | 1.00 |
| Sodium sulfite | — | — | — | — | — | 0.20 |
| Hexetidine | — | 0.10 | 0.10 | — | — | 0.10 |
| Formic acid | — | — | — | — | — | 0.50 |
| Phenoxyisopropanol | 1.00 | — | — | — | — | 1.00 |
| Undecylenic acid | — | — | — | — | — | 0.20 |
| Chloroxylenol | — | — | — | — | — | 0.50 |
| Benzyl alcohol | — | — | — | — | — | 1.00 |
| Chlorphenesin | — | — | 0.30 | — | — | 0.30 |
| KM ¹⁾ | — | — | — | 0.3 | 1.2 | — |
| Panthenol | 0.08 | 0.08 | 0.08 | 0.08 | 0.08 | 0.08 |
| Tocopheryl acetate | 0.05 | 0.05 | 0.05 | 0.05 | 0.05 | 0.05 |
| Sorbitan oleate | 0.02 | 0.02 | 0.02 | 0.02 | 0.02 | 0.02 |
| Caprylyl/capryl glucoside | 0.01 | 0.01 | 0.01 | 0.01 | 0.01 | 0.01 |
| Aqua | up to | up to | up to | up to | up to | up to |
| | 100 | 100 | 100 | 100 | 100 | 100 |

¹⁾ Preservative mixture selected from at least one of the preservative mixtures KM1 to KM157 listed above.

Cleansing emulsion (data in wt %)

| Raw material | 6.1 | 6.2 | 6.3 | 6.4 | 6.5 | 6.6 |
|---|---|---|---|---|---|---|
| Glycerin | 5.01 | 5.01 | 5.01 | 5.01 | 5.01 | 5.01 |
| Sorbitol | 2.10 | 2.10 | 2.10 | 2.10 | 2.10 | 2.10 |
| Hexyl laurate | 2.00 | 2.00 | 2.00 | 2.00 | 2.00 | 2.00 |
| Ethylhexyl palmitate | 2.00 | 2.00 | 2.00 | 2.00 | 2.00 | 2.00 |
| Hydroxyethyl urea | 1.34 | 1.34 | 1.34 | 1.34 | 1.34 | 1.34 |
| Cetearyl alcohol | 1.00 | 1.00 | 1.00 | 1.00 | 1.00 | 1.00 |
| Aluminum starch | 0.93 | 0.93 | 0.93 | 0.93 | 0.93 | 0.93 |
| octenylsuccinate | | | | | | |
| Glyceryl stearate | 0.50 | 0.50 | 0.50 | 0.50 | 0.50 | 0.50 |
| Potassium cetyl phosphate | 0.31 | 0.31 | 0.31 | 0.31 | 0.31 | 0.31 |
| Carbomer | 0.30 | 0.30 | 0.30 | 0.30 | 0.30 | 0.30 |
| Perfume | 0.19 | 0.19 | 0.19 | 0.19 | 0.19 | 0.19 |
| Hydrogenated palm | 0.19 | 0.19 | 0.19 | 0.19 | 0.19 | 0.19 |
| glycerides | | | | | | |
| Sodium acrylate/sodium | 0.19 | 0.19 | 0.19 | 0.19 | 0.19 | 0.19 |
| acryloyldimethyl taurate | | | | | | |
| copolymer | | | | | | |
| Ethyl lauroyl arginate*HCl | — | — | 0.40 | — | — | 0.40 |
| Piroctone olamine | — | — | — | — | — | 1.00 |
| Sodium sulfite | — | — | — | — | — | 0.20 |
| Hexetidine | — | 0.10 | 0.10 | — | — | 0.10 |
| Formic acid | 0.50 | 0.50 | — | — | — | 0.50 |
| Phenoxyisopropanol | 1.00 | — | — | — | — | 1.00 |
| Undecylenic acid | — | — | — | — | — | 0.20 |
| Chloroxylenol | — | — | — | — | — | 0.50 |
| Benzyl alcohol | — | — | — | — | — | 1.00 |
| Chlorphenesin | — | — | — | — | — | 0.30 |
| KM ¹⁾ | — | — | — | 0.9 | 0.25 | — |
| Panthenol | 0.08 | 0.08 | 0.08 | 0.08 | 0.08 | 0.08 |
| Urea | 0.08 | 0.08 | 0.08 | 0.08 | 0.08 | 0.08 |
| Tocopheryl acetate | 0.05 | 0.05 | 0.05 | 0.05 | 0.05 | 0.05 |
| Sorbitan oleate | 0.02 | 0.02 | 0.02 | 0.02 | 0.02 | 0.02 |
| Caprylyl/capryl glucoside | 0.01 | 0.01 | 0.01 | 0.01 | 0.01 | 0.01 |
| Aqua | up to | up to | up to | up to | up to | up to |
| | 100 | 100 | 100 | 100 | 100 | 100 |

¹⁾ Preservative mixture selected from at least one of the preservative mixtures KM1 to KM157 listed above.

Cleansing milk (data in wt %)

| Raw material | 7.1 | 7.2 | 7.3 | 7.4 | 7.5 | 7.6 |
|---|---|---|---|---|---|---|
| Helianthus annuus seed oil | 5.00 | 5.00 | 5.00 | 5.00 | 5.00 | 5.00 |
| Isopropyl palmitate | 4.98 | 4.98 | 4.98 | 4.98 | 4.98 | 4.98 |
| Rosa damascena flower water | 4.97 | 4.97 | 4.97 | 4.97 | 4.97 | 4.97 |
| Ethyl lauroyl arginate*HCl | — | — | — | — | — | 0.40 |
| Piroctone olamine | 0.5 | — | — | — | — | 1.00 |
| Sodium sulfite | — | — | 0.20 | — | — | 0.20 |
| Hexetidine | — | — | — | — | — | 0.10 |
| Formic acid | — | — | — | — | — | 0.50 |
| Phenoxyisopropanol | 1.00 | — | — | — | — | 1.00 |
| Undecylenic acid | — | — | — | — | — | 0.20 |
| Chloroxylenol | — | 0.50 | — | — | — | 0.50 |
| Benzyl alcohol | — | 1.00 | — | — | — | 1.00 |
| Chlorphenesin | — | — | 0.30 | — | — | 0.30 |
| KM ¹⁾ | — | — | — | 0.7 | 0.01 | — |
| Cetearyl alcohol | 2.50 | 2.50 | 2.50 | 2.50 | 2.50 | 2.50 |
| Cocoglycerides | 2.00 | 2.00 | 2.00 | 2.00 | 2.00 | 2.00 |
| Laurus nobilis leaf extract | 1.99 | 1.99 | 1.99 | 1.99 | 1.99 | 1.99 |
| Hydrogenated vegetable oil | 1.00 | 1.00 | 1.00 | 1.00 | 1.00 | 1.00 |
| Xanthan gum | 1.00 | 1.00 | 1.00 | 1.00 | 1.00 | 1.00 |
| Lauryl glucoside | 0.95 | 0.95 | 0.95 | 0.95 | 0.95 | 0.95 |
| Polyglyceryl-2 | 0.95 | 0.95 | 0.95 | 0.95 | 0.95 | 0.95 |
| dipolyhydroxystearate | | | | | | |
| Panthenol | 0.50 | 0.50 | 0.50 | 0.50 | 0.50 | 0.50 |
| Citric acid | 0.21 | 0.21 | 0.21 | 0.21 | 0.21 | 0.21 |
| Sodium stearoyl glutamate | 0.10 | 0.10 | 0.10 | 0.10 | 0.10 | 0.10 |
| Tocopheryl acetate | 0.09 | 0.09 | 0.09 | 0.09 | 0.09 | 0.09 |
| Aqua | up to | up to | up to | up to | up to | up to |
| | 100 | 100 | 100 | 100 | 100 | 100 |

¹⁾ Preservative mixture selected from at least one of the preservative mixtures KM1 to KM157 listed above.

Impregnation solution for cleansing wipes (data in wt %)

| Raw material | 8.1 | 8.2 | 8.3 | 8.4 | 8.5 | 8.6 |
|---|---|---|---|---|---|---|
| Propylheptyl caprylate | 5.00 | 5.00 | 5.00 | 5.00 | 5.00 | 5.00 |
| Glycerin | 2.72 | 2.72 | 2.72 | 2.72 | 2.72 | 2.72 |
| Isopropyl palmitate | 2.00 | 2.00 | 2.00 | 2.00 | 2.00 | 2.00 |
| Lauryl glucoside | 1.36 | 1.36 | 1.36 | 1.36 | 1.36 | 1.36 |
| Polyglyceryl-2 | 1.36 | 1.36 | 1.36 | 1.36 | 1.36 | 1.36 |
| dipolyhydroxystearate | | | | | | |
| Caprylic/capric triglyceride | 1.00 | 1.00 | 1.00 | 1.00 | 1.00 | 1.00 |
| Phenoxyethanol | 0.58 | 0.58 | 0.58 | 0.58 | 0.58 | 0.58 |
| Sodium polyacrylate | 0.35 | 0.35 | 0.35 | 0.35 | 0.35 | 0.35 |
| Perfume | 0.29 | 0.29 | 0.29 | 0.29 | 0.29 | 0.29 |
| Ethyl lauroyl arginate*HCl | — | — | 0.40 | — | — | 0.40 |
| Piroctone olamine | 1.0 | — | — | — | — | 1.00 |
| Sodium sulfite | — | — | — | — | — | 0.20 |
| Hexetidine | 0.1 | 0.10 | — | — | — | 0.10 |
| Formic acid | — | — | — | — | — | 0.50 |
| Phenoxyisopropanol | — | — | — | — | — | 1.00 |
| Undecylenic acid | — | 0.20 | 0.20 | — | — | 0.20 |
| Chloroxylenol | — | — | — | — | — | 0.50 |
| Benzyl alcohol | — | — | — | — | — | 1.00 |
| Chlorphenesin | — | — | — | — | — | 0.30 |
| KM ¹⁾ | — | — | — | 1.8 | 0.09 | — |
| Argania spinosa kernel oil | 0.10 | 0.10 | 0.10 | 0.10 | 0.10 | 0.10 |
| Citric acid | 0.03 | 0.03 | 0.03 | 0.03 | 0.03 | 0.03 |
| Aqua | up to | up to | up to | up to | up to | up to |
| | 100 | 100 | 100 | 100 | 100 | 100 |

¹⁾ Preservative mixture selected from at least one of the preservative mixtures KM1 to KM157 listed above.

Impregnation solutions for cleansing wipes (data in wt %)

| Raw material | 9.1 | 9.2 | 9.3 | 9.4 | 9.5 | 9.6 |
|---|---|---|---|---|---|---|
| Glycerin | 4.49 | 4.49 | 4.49 | 4.49 | 4.49 | 4.49 |
| Cetearyl isononanoate | 1.13 | 1.13 | 1.13 | 1.13 | 1.13 | 1.13 |
| Ceteareth-20 | 0.56 | 0.56 | 0.56 | 0.56 | 0.56 | 0.56 |
| Cetearyl alcohol | 0.56 | 0.56 | 0.56 | 0.56 | 0.56 | 0.56 |
| Potassium sorbate | 0.30 | 0.30 | 0.30 | 0.30 | 0.30 | 0.30 |
| Glyceryl stearate | 0.19 | 0.19 | 0.19 | 0.19 | 0.19 | 0.19 |
| Perfume | 0.17 | 0.17 | 0.17 | 0.17 | 0.17 | 0.17 |
| Ethyl lauroyl | — | 0.40 | — | — | — | 0.40 |
| arginate*HCl | | | | | | |
| Piroctone olamine | — | — | — | — | — | 1.00 |
| Sodium sulfite | — | — | 0.20 | — | — | 0.20 |
| Hexetidine | — | — | — | — | — | 0.10 |
| Formic acid | — | — | 0.50 | — | — | 0.50 |
| Phenoxyisopropanol | 1.0 | — | — | — | — | 1.00 |
| Undecylenic acid | — | — | 0.20 | — | — | 0.20 |
| Chloroxylenol | 0.50 | — | — | — | — | 0.50 |
| Benzyl alcohol | — | — | — | — | — | 1.00 |
| Chlorphenesin | — | 0.30 | — | — | — | 0.30 |
| KM ¹⁾ | — | — | — | 0.001 | 0.4 | — |
| Ceteareth-12 | 0.04 | 0.04 | 0.04 | 0.04 | 0.04 | 0.04 |
| Cetyl palmitate | 0.04 | 0.04 | 0.04 | 0.04 | 0.04 | 0.04 |
| Aqua | up to | up to | up to | up to | up to | up to |
| | 100 | 100 | 100 | 100 | 100 | 100 |

¹⁾ Preservative mixture selected from at least one of the preservative mixtures KM1 to KM157 listed above.

Impregnation solution for cleansing wipes (data in wt %)

| Raw material | 10.1 | 10.2 | 10.3 | 10.4 | 10.5 | 10.6 |
|---|---|---|---|---|---|---|
| Glycerin | 4.28 | 4.28 | 4.28 | 4.28 | 4.28 | 4.28 |
| Paraffinum liquidum | 4.00 | 4.00 | 4.00 | 4.00 | 4.00 | 4.00 |
| PPG-15 stearyl ether | 2.50 | 2.50 | 2.50 | 2.50 | 2.50 | 2.50 |
| Steareth-10 | 0.75 | 0.75 | 0.75 | 0.75 | 0.75 | 0.75 |
| Glyceryl stearate | 0.47 | 0.47 | 0.47 | 0.47 | 0.47 | 0.47 |
| PEG-30 STEARATE | 0.28 | 0.28 | 0.28 | 0.28 | 0.28 | 0.28 |
| Perfume | 0.17 | 0.17 | 0.17 | 0.17 | 0.17 | 0.17 |
| Ethyl lauroyl | — | 0.40 | — | — | — | 0.40 |
| arginate*HCl | | | | | | |
| Piroctone olamine | 0.60 | — | — | — | — | 1.00 |
| Sodium sulfite | — | — | 0.20 | — | — | 0.20 |
| Hexetidine | 0.10 | — | — | — | — | 0.10 |
| Formic acid | — | — | 0.50 | — | — | 0.50 |
| Phenoxyisopropanol | — | — | — | — | — | 1.00 |
| Undecylenic acid | — | — | 0.20 | — | — | 0.20 |
| Chloroxylenol | — | — | — | — | — | 0.50 |
| Benzyl alcohol | — | — | — | — | — | 1.00 |
| Chlorphenesin | — | 0.30 | — | — | — | 0.30 |
| KM ¹⁾ | — | — | — | 0.001 | 0.4 | — |
| Aqua | up to | up to | up to | up to | up to | up to |
| | 100 | 100 | 100 | 100 | 100 | 100 |

¹⁾ Preservative mixture selected from at least one of the preservative mixtures KM1 to KM157 listed above.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent, including, in a cosmetically acceptable carrier,
a) at least one emulsifier,
b) at least one compound selected from the group of oils, waxes, esters or mixtures thereof,
c) at least one preservative mixture selected from the group consisting of
chloroxylenol and phenoxyisopropanol,
undecylenic acid and formic acid,
phenoxyisopropanol and piroctone olamine,
phenoxyisopropanol and formic acid,
sulfite(s) and hexetidine,
ethyl lauroyl arginate and formic acid,
ethyl lauroyl arginate and chloroxylenol,
hexetidine and benzyl alcohol,
hexetidine and chloroxylenol,
hexetidine and piroctone olamine,
hexetidine and chlorphenesin,
hexetidine and formic acid, and
mixtures of such combinations.

2. The cosmetic agent as set forth in claim 1, wherein the at least one emulsifier is included at a concentration of 0.1 to 40 wt.% with respect to the cosmetic agent's total weight.

3. The cosmetic agent as set forth in claim 1, wherein the at least one compound selected from the group of oils, waxes, esters, or mixtures thereof cosmetic agent is include at a concentration of 0.1 to 50 wt % with respect to the cosmetic agent's total weight.

4. The cosmetic agent as set forth in claim 1, wherein a weight ratio of the first preservative to the second preservative in the preservative mixture c) ranges from 10:1 to 1:10.

5. The cosmetic agent as set forth in claim 1, wherein the at least one preservative mixture is included at a concentration of 0.001 to 10 wt %, with respect to the cosmetic agent's total weight.

6. A cleansing composition as set forth in claim 1, further including at least one additional preservative selected from the group consisting of benzyl alcohol(s), chloroxylenol, phenoxyisopropanol, undecylenic acid, formic acid, piroctone olamine, sulfite(s), hexetidine, ethyl lauroyl arginate, and chlorphenesin, provided that the cleansing composition includes at least 3 mutually different preservatives.

7. The cleansing composition as set forth in claim 1, further including at least two additional preservatives selected from the group consisting of benzyl alcohol(s), chloroxylenol, phenoxyisopropanol, undecylenic acid, formic acid, piroctone olamine, sulfite(s), hexetidine, ethyl lauroyl arginate, and chlorphenesin, provided that the cleansing composition includes at least 4 mutually different preservatives.

8. The cleansing composition as set forth in claim 1, further including at least six additional preservatives selected from the group consisting of benzyl alcohol(s), chloroxylenol, phenoxyisopropanol, undecylenic acid, formic acid, piroctone olamine, sulfite(s), hexetidine, ethyl lauroyl arginate, and chlorphenesin, provided that the cleansing composition includes at least 8 mutually different preservatives.

9. The cleansing composition as set forth in claim 6, wherein the at least one additional preservative is included at a concentration of 0.1 to 10 wt % with respect to the cosmetic agent's total weight.

10. A method of preserving a cosmetic cleansing composition in the form of an emulsion, the method comprising:
including in the cosmetic cleansing composition at least one preservative mixture selected from the group consisting of chloroxylenol and phenoxyisopropanol, undecylenic acid and formic acid, phenoxyisopropanol and piroctone olamine, phenoxyisopropanol and formic acid, sulfite(s) and hexetidine, ethyl lauroyl arginate and formic acid, ethyl lauroyl arginate and chloroxylenol, hexetidine and benzyl alcohol, hexetidine and chloroxylenol, hexetidine and piroctone olamine, hexetidine and chlorphenesin, hexetidine and formic acid, and mixtures of such combinations.
